(19) United States
(12) Patent Application Publication
(10) Pub. No.: US 2019/0164638 A1
(43) Pub. Date: May 30, 2019

(54) AN ORDERING SYSTEM

(71) Applicant: LINDE AKTIENGESELLSCHAFT, Munich (DE)

(72) Inventors: Brian JACOBSEN, Horsham, West Sussex (GB); Mandip THIND, Southall (GB)

(73) Assignee: LINDE AKTIENGESELLSCHAFT, Munich (DE)

(21) Appl. No.: 16/315,248

(22) PCT Filed: Jul. 6, 2017
(86) PCT No.: PCT/EP2017/067024

§ 371 Date: Jan. 4, 2019

(30) Foreign Application Priority Data
Jul. 7, 2016 (GB) 1611858.0

(51) Int. Cl. G16H 20/10; G16H 40/20; G06Q 10/08; G06Q 30/06

(52) U.S. Cl. CPC G16H 20/10; G06Q 30/0635; G06Q 10/087; G16H 40/20

(57) ABSTRACT

An ordering system (10) comprising a database (60) for storing customer specific data (CSD) and associated pre-defined product specific data (PSD), a first transceiver (14) connected to the database (60) and operable to transmit and receive data to and from the database (60), a customer located transceiver (36) operable to transmit and receive a predefined product order (PO) and customer specific data (CSD) to and from the first transceiver (14), and a comparison module (42) operable to compare a received predefined product order (PO) with the pre-defined product specific data (PSD) and the customer specific data (CSD) and cause the creation of a verification signal (VS) upon receipt of a product order (PO) which matches with the pre-defined product specific data (PSD) and the customer specific data (CSD).

The present invention relates to an ordering system, in particular to an ordering system for the supply of medical products in the form of gas supply systems such as gas cylinders containing clinical gases, for example, oxygen, nitrous oxide, Xenon, nitric oxide, helium and mixtures thereof, liquid oxygen, and concentrators. The present invention also relates to consumables for use with such gas supply systems.

For many medical conditions, patients require a supply of gas such as oxygen from a gas cylinder. In addition, consumables associated with that gas supply, such as face masks and filters, need replacing at designated times to ensure the safe supply of gas to the patient. Collectively, the gas cylinders and consumables are referred to as product.

Typically, patients requiring the supply of gas are treated in a medical environment such as a hospital, however, patients can also be treated in non-medical environments such as at the patient's home.

It is important that the gas in the gas cylinders does not run out to ensure the patient always has a gas supply, and furthermore that the consumables associated with supplying the gas to the patient are replaced at the designated time.

In a medical environment such as a hospital, the cylinder gas levels and consumables are regularly monitored by medical professionals, and a dedicated ordering system is in place to replace gas cylinders and consumables as required.

In contrast, patient's undergoing treatment in non-medical locations are more vulnerable to gas cylinders not being replaced before they become empty as the monitoring of the level of gas in the gas cylinder less frequent and reliable compared to a medical environment where trained medical staff are present. In addition, the time to replace gas cylinders once identified as being empty is greater as the replacement gas cylinders will need to be transported from a storage location which is remote from the non-medical location. This is a greater concern for patients who are in need of a constant gas supply where the consequences of running out of gas is more serious.

Often, patients do not place orders for consumables at the designated time because they are either unaware they require replacing or simply forget to place the order, the result being that those consumables are not replaced in a timely manner which can result in the consumables not functioning correctly and also reduced revenue from the supplier of those consumables.

Repeat ordering of gas cylinders can also be difficult to manage logistically if the patient is not available to confirm the order, this being further complicated in some countries such as the USA where the supply of medical products is subject to regulations which prevents the delivery of the new gas cylinder unless there is an order from the patient.

A more reliable ordering system for gas cylinders and other gas supply systems and associated consumables is therefore desired.

Thus, according to the present invention there is provided an ordering system comprising a database for storing customer specific data (CSD) and associated pre-defined product specific data (PSD), a first transceiver operable to transmit and receive data to and from the database, a second customer located transceiver operable to transmit and receive a pre-defined product order (PO) and the customer specific data (CSD) to and from the first transceiver, and a comparison system operable to compare the received pre-defined product order (PO) with the pre-defined product specific data (PSD) and the customer specific data (CSD) and cause the creation of a verification signal (VS) upon receipt of a is product order (PO) which matches with the pre-defined product specific data (PSD) and the customer specific data (CSD).

The customer is able to initiate a product order (PO) from the customer located transceiver which is then compared to the product specific data (PSD) and the customer specific data (CSD) to ensure that the customer is entitled to the product. In the case of the customer being a patient, entitlement is typically based on the patient's insurance plan associated with the product specific data (PSD) and the customer specific data (CSD). If there is a match, i.e. if the insurance plan entitles the patient to the product order (PO), then a verification signal (VS) is created enabling the product order (PO) to be fulfilled. This has the advantage that the patient is able to initiate a product order (PO) from where he or she is located which is easier for the patient, and in some countries, is necessary to comply with regulations for the delivery of medical products.

The product order (PO) can be a gas supply system such as a gas cylinder, liquid oxygen, or a concentrator, as well as the consumables associated with the gas supply system. Typically patients requiring long-term oxygen therapy use liquid oxygen and concentrators rather than gas cylinders.

Preferably the ordering system comprises a message display on the second customer located transceiver for indicating future product ordering information (FPOI) based on the product specific data (PSD) and/or product order status. This has the advantage of giving the patient a visual indication that a product order (PO) can be initiated, and confirmation if an order has been successfully fulfilled.

Preferably the customer located transceiver is configured for receiving product specific data (PSD) in advance of that required by a patient for the treatment thereof and the message display is configured for displaying the advanced product specific data (APSD) in advance of a requirement therefor such as to allow said patient to order product required for treatment purposes in advance of a specific need therefor. Advantageously this enables the patient to be aware of, and enable the product order to be initiated before the product is required by the patient.

The product specific data (PSD) can comprise multiple products for different patient treatments, and each of the products can have different consumption rates and/or expiry dates.

The pre-defined advanced product specific data (APSD) can include a listing of multiple products required individually or in combination with each other within a pre-defined is window of use (WOU).

The second patient located transceiver can include a memory for storing information relating to the pre-defined advanced product specific data (APSD).

The ordering system can include an inventory management system for receiving inventory data associated with the product specific data (PSD) from a supplier thereof and being connected to the database for the exchange of information therebetween and wherein said ordering system includes an inventory checker for periodically checking that products identified in the advanced product specific data (APSD) are in stock and available for dispatch. This enables checks to be made to stock levels of products in advance of the order needing to be fulfilled.

The ordering system can include a stock ordering system for receiving data relating to stock levels of products included in the advanced product specific data (APSD) and for receiving advanced product specific data (APSD) and for determining if new stock is needed and for initiating the ordering thereof if stock is needed. This ensures that product can be ordered to enable fulfillment of an order placed by the patient.

The ordering system can include a patient specific database for receiving pre-defined product specific data (PPSD) specific to a given patient having a specific associated second patient located transceiver. This enables confidential patient data to be stored in a different database to the data of other patients, increasing security, and complying with confidentiality regulations in certain countries.

The patient specific database can also be amendable to ensure the advanced product specific data (APSD) stored on the patient located transceiver matches the pre-defined product specific data (PPSD) stored on the patient specific database. In one embodiment the ordering system includes an access device for accessing the amendable patient specific database and updating if there is a mismatch. This ensures the product specific data stored on the patient specific database is up-to-date.

The ordering system can also include a message updater operable to periodically check the advanced product specific data (APSD) stored on the customer located transceiver against current pre-defined product specific data (CPSD) stored on the amendable patient specific database and for initiating an updated transmission of advanced product specific data (APSD) to the second customer located transceiver if there is a miss-match between the advanced product specific data (APSD) stored on the second customer located is transceiver and the current pre-defined product specific data (PPSD) stored on the patient specific amendable database (60). This ensures the product specific data stored on the customer located transceiver is up-to-date.

The comparison module can be configured for transmission of a verification of order message (VOM) to the customer located transceiver upon identification of a match between the product order (PO) and the product specific data (PSD). If there is a negative or no match between the product order (PO) and the product specific data (PSD), a failure of order message (FOM) is transmitted to the customer located transceiver.

The verification of order message (VOM) can be displayed on a visual display included on the customer located transceiver, the display configured to display a first colour upon receipt of a failure order message (FOM) and a second colour upon receipt of a verification order message (VOM).

The first transceiver is operable to transmit a modified pre-defined advanced product specific data (APSD) to the customer located transceiver after an earlier pre-defined product order (PO) has been accepted for delivery to ensure the advanced product specific data (APSD) is up-to-date.

The ordering system can also include a patient credit status checking module for checking the credit status of the patient prior to the instruction to dispatch any product to the patient.

The invention will now be described by way of example only with reference to the accompanying drawings, in which:

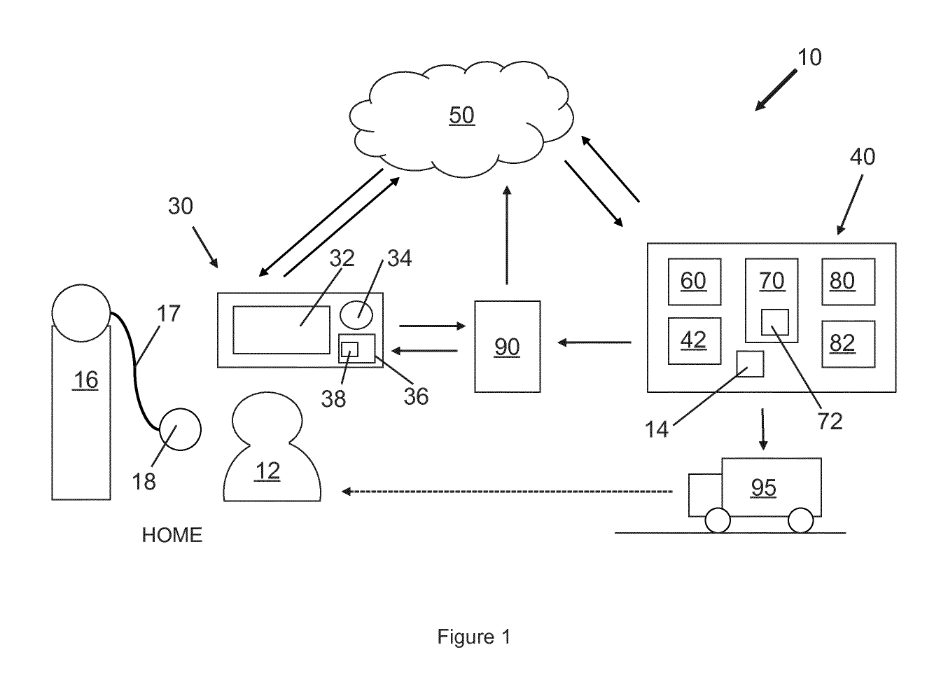

FIG. 1 is a schematic view of the ordering system of the present invention.

In FIG. 1, the ordering system 10 includes a button module 30, a central management system 40, and a cloud-based button message server 50.

The central management system 40 includes a database in the form of an amendable patient specific database 60, an inventory management system 70 and a stock ordering system 80.

The patient specific database 60 stores customer specific data (CSD) relating to a specific consumer, which in this embodiment is a patient 12, and pre-defined product specific data (PSD), which in this embodiment relates to an insurance plan of the patient 12, and determines which products associated with a product order (PO) the patient 12 is entitled to.

The central management system 40 also includes a comparison module 42 for comparing the product order (PO) with the product specific data (PSD) and the patient specific data (CSD) to see if there is a match and generating a verification signal (VS) in the form of a verification of order message (VOM) if there is a match, or a failure signal (FS) in the form of a failure of order message (FOM) if there is not a match. The failure or verification messages (FOM,VOM) are transmitted from the central management system 40 to the button module 30 via the first transceiver 14.

The central management system 40 is connected to a first transceiver 14 which enables data to be transmitted between, and to and from the patient specific database 60, the inventory management system 70 and the stock ordering system 80.

The patient 12 is located at home and receives medical treatment in the form of a gas supply, in this embodiment, the supply of oxygen from a gas cylinder 16 via face mask 18. The face mask 18 is connected to the gas cylinder via tube 17 and includes a filter (not shown) and a sponge (not shown). The face mask 18, filter, and sponge are considered to be consumables and the consumables and the gas cylinders are considered to be products associated with a product order (PO).

The button module 30 is located in the home of the patient 12 and includes a visual display 32, a button 34, and a Wi-Fi transceiver 36. The transceiver 36 includes a storage memory 38. The button module 30 is configured to receive product specific data (PSD) via the transceiver 36 and display the status of one or more product orders (POs) relating to products having different consumption rates and/or expiry dates associated with the product specific data (PSD), for example, future product ordering information (FPOI), and/or verification of order message (VOM) or failure of order message (FOM), on the visual display 32. The button module 30 is also configured to transmit data to the central management system 40 via the cloud-based button message server 50.

The inventory management system 70 is connected to the patient specific database 60 and includes an inventory checker 72 which can periodically check products required to fulfill orders associated with the product specific data (PSD) are in stock with the supplier of those products, and available for dispatch. If the products are not in stock, the stock ordering system 70 initiates an order with the supplier to ensure that when the product order (PO) needs to be initiated, there is sufficient stock.

The ordering system 10 also includes an access device in the form of a technician's mobile phone 90 which is required to initially set-up the button module 30 with the central management system 40.

In the above embodiment, the button module 30 is a physical device. In an alternative embodiment the features of the button module can be incorporated into computer software, for example in an app, with the button replaced by a virtual button and the visual display utilising the screen of a mobile device, for example a mobile telephone or tablet, on which the computer software is installed. This has the advantage that the patient will be able to initiate orders without relying on a physical button module, and whilst the button module is mobile, incorporating the features into computer software installed on a mobile device increases the likelihood of the patient being contactable and therefore the likelihood that product orders can be processed.

In the above embodiment the advanced product specific data (APSD) is stored in the memory 38 of the button module 30. In an alternative embodiment, the advanced product specific data (APSD) can be stored in the central patient specific database 60.

In the above embodiments, data specific to a single patient is stored on a patient specific database (CSD). Alternatively, data associated with more than one patient can be stored in a single database.

The ordering system operates as follows:

Initially, the button module 30 and the button message server 50 are configured so that they recognise each other as follows:

A technician signs into the software application installed on their mobile phone 90 and retrieves the patient's account details.

The technician then connects the mobile phone 90 to the button module 30 using the Wi-Fi transceiver 36 as an access point (AP). A password is required to prevent an unauthorised connection and access to sensitive patient data.

Once the password is confirmed, the button module 30 sends a unique MAC address to the mobile phone 90. After the mobile phone receives the MAC address of the button module 30, the connection is broken.

The patient's account details are then identified from the unique MAC address, and the mobile phone 90 retrieves the patient's product specific data (PSD) from the central management system 40. The patient's product specific data is then transmitted to the button message server 50 where it is stored.

Once configured, and at a time according to the pre-defined product specific data (PSD), the central management system 40 sends a signal to the button module 30 via the button message server 50 to indicate that a product order (PO) is ready to be processed.

The patient 12 is alerted that the product order (PO) is ready to be processed by a visual signal in the form of a flashing LED on the message display 32 and optionally an audible signal.

On hearing the audible signal and/or seeing the flashing LED, the patient 12 will press the button 34 to confirm the product order (PO) is to be processed. It will be appreciated that the visible signal on the button, specifically, the flashing LED indicating a product order (PO) is ready to be processed, will remain until the button is pressed by the patient. The button can be configured to repeat the audible signals at regular intervals until the button is pressed to ensure the patient is alerted to the requirement to confirm the processing of the order.

On being pressed, the button module 30 wakes up and establishes a Wi-Fi connection with the configured access point (AP) and sends an MQTT message containing it's MAC address and battery level to the cloud-based button message server 50.

The button message server 50 determines the patient account ID (PID) and the pre-defined product order (PO), both of which are stored on the button message server 12, from the received MAC address of the button module 50, and sends both the product order (PO) and the patient account ID (PID) to the comparison module 42.

The comparison module 42 identifies the patient specific data (CSD) from the linked patient account ID (PID), and the product specific data (PSD) associated with the patient specific data (CSD). The product order (PO) is then compared to the product specific data (PSD), and if there is a match, the comparison system generates a verification signal (VS).

The verification signal (VS) is then sent to the button module 30 in the form of a verification of order message (VOM) via the button message server 50 to indicate that the product order (PO) has been successful, with a visual indication of the successful order shown on the message display 32 in the form of a green coloured light.

If there is no match between the product order (PO) and the product specific data (PSD) then a failure of order message (FOM) is sent to the button module 30, with a visual indication of the failed order shown on the message display 32 in the form of a red coloured light.

At the same time as sending confirmation of the successful order, the central management system 40 also sends details of the next product order (PO) to the button module 30 via the button message server 50, the product order (PO) details then being indicated on the message display 32.

If the order is successful, then the central management system 40 instructs the delivery of the product or products associated with the product order (PO) to the patient by transport such as a truck 95 (indicated by a broken line in FIG. 1).

The button module 30 will also periodically check (via a message updater 82) that the advanced product specific data (APSD) stored on the customer located transceiver (36) is the same as that stored on the amendable patient specific database (60). If that is not the case, then an updated advanced product specific data (APSD) is transmitted to the customer located transceiver (36).

The inventory management system (70) and stock inventory system (80) ensure that there is sufficient stock to fulfill orders by periodically checking that products identified in the advanced product specific data (APSD) are in stock and available for dispatch, and initiating the ordering thereof if stock is needed to fulfill the order.

The above embodiments describe an ordering system used with a patient located at home. The ordering system can also be used in a medical environment such as a hospital where the button module is associated with gas cylinders in a ward, and medical staff can initiate the replacement of empty gas cylinders by pressing the button on the button module in the same way as the patient initiates a product order in the embodiments above.

1. An ordering system (10) comprising:
a database (60) for storing customer specific data (CSD) and associated pre-defined product specific data (PSD),
a first transceiver (14) connected to the database (60) and operable to transmit and receive data to and from the database (60),
a customer located transceiver (36) operable to transmit and receive a pre-defined product order (PO) and customer specific data (CSD) to and from the first transceiver (14), and
a comparison module (42) operable to compare a received pre-defined product order (PO) with the pre-defined product specific data (PSD) and the customer specific data (CSD) and cause the creation of a verification signal (VS) upon receipt of a product order (PO) which matches with the pre-defined product specific data (PSD) and the customer specific data (CSD).

2. An ordering system (10) according to claim 1 including a message display (32) on the customer located transceiver (36) operable to display future product ordering information (FPOI) based on the pre-defined product specific information (PSD) and/or to display a visual indication of the status of a product order (PO).

3. An ordering system (10) according to claim 2 in which the customer located transceiver (36) is configured to receive product specific data (PSD) in advance of that required by a patient for the treatment thereof, and in which the message display (32) is configured to display the advanced product specific data (APSD) in advance of a requirement therefor so as to allow the patient to order product required for treatment purposes in advance of a specific need therefor.

4. An ordering system (10) according to claim 3 in which the pre-defined product specific data (PSD) comprises multiple products required for patient treatment.

5. An ordering system (10) according to claim 4 in which the pre-defined product specific data (PSD) comprises multiple products having multiple different consumption rates and/or expiry dates and wherein said pre-defined advanced product specific data (APSD) includes a listing of multiple products required individually or in combination with each other within a pre-defined window of use (WOU).

6. An ordering system (10) according to claim 3 in which the customer located transceiver (36) includes a memory (38) for storing information relating to pre-defined advanced product specific data (APSD).

7. An ordering system (10) as claimed in claim 3 including an inventory management system (70) for receiving inventory data associated with the product specific data (PSD) from a supplier thereof and being connected to the database (60) for the exchange of information therebetween and wherein said ordering system (10) includes an inventory checker (72) for periodically checking that products identified in the advanced product specific data (APSD) are in stock and available for dispatch.

8. An ordering system (10) according to claim 7 including a stock ordering system (80) for receiving data relating to stock levels of products included in the advanced product specific data (APSD) and for determining if new stock is needed and for initiating the ordering thereof if stock is needed to fulfill an order relating to advanced, product specific data (APSD).

9. An ordering system (10) according to claim 1 in which the database (60) is a patient specific database (60) for receiving pre-defined product specific data (PSD) specific to a given patient having a specific associated customer located transceiver (36).

10. An ordering system (10) according to claim 9 in which the patient specific database (60) is an amendable patient specific database (60), the ordering system further comprising an access device (90) for accessing and updating the amendable patient specific database (60).

11. An ordering system (10) according to claim 10 including a message updater (82) operable to periodically check the advanced product specific data (APSD) stored on the customer located transceiver (36) against current pre-defined product specific data (CPSD) stored on the amendable patient specific database (60) and for initiating the transmission of an updated transmission of advanced product specific data (APSD) to the customer located transceiver (36) if there is a mismatch between the advanced product specific data (APSD) stored on the customer located transceiver (36) and the current pre-defined product specific data (PPSD) stored on the amendable database (60).

12. An ordering system (10) according to claim 1 in which the comparison module (42) is configured for transmission of a verification of order message (VOM) to the customer located transceiver (16) upon identification of a match between the product order (PO) and the product specific data (PSD).

13. An ordering system (10) according to claim 12 in which the comparison module (42) is configured for transmission of a failure of order message (FOM) to the customer located transceiver (16) upon identification of a negative match between the product order (PO) and the product specific data (PSD).

14. An ordering system (10) according to claim 12 in which the verification of order message (VOM) or failure of order message (FOM) is displayable on the message display (32).

15. An ordering system (10) according to claim 14 in which the message display (32) includes a coloured light configured to display a first colour upon receipt of a failure order message (FOM) and a second colour upon receipt of a verification order message (VOM).

16. An ordering system (10) according to claim 1 in which the customer located transceiver (16) is a patient located transceiver (16) located with a patient.

17. An ordering system (10) according to claim 16 in which the patient is located at home.

18. An ordering system (10) according to claim 16 including a patient credit status checking module (80) for checking the credit status of the patient prior to the instruction to dispatch any product to said patient.

19. An ordering system (10) according to claim 6 in which the first transceiver (16) is operable to transmit a modified pre-defined advanced product specific data (APSD) to the second customer located transceiver (36) after an earlier pre-defined product order (PO) has been accepted for delivery.

20. An ordering system (10) according to claim 1 in which the product specific data (PSD) is a health insurance plan.

21. An ordering system (10) according to claim 1 in which the product order (PO) relates to a medical product.

22. An ordering system (10) according to claim 21 in which the medical product is a gas supply and/or consumables associated with the gas supply.

23. An ordering system (10) according to claim 22 in which the gas supply is a gas cylinder.
